SEQUENCE LISTING (10 sequences)

SEQ ID NO: 1 - DNA, 20 bp, Homo sapiens
  misc_feature 1..20: note = beta-actin (sense)
  source 1..20: mol_type = genomic DNA
accttctacaatgagctgcg

SEQ ID NO: 2 - DNA, 21 bp, Homo sapiens
  misc_feature 1..21: note = beta-actin (antisense)
  source 1..21: mol_type = genomic DNA
cctggatagcaacgtacatgg

SEQ ID NO: 3 - DNA, 18 bp, Homo sapiens
  misc_feature 1..18: note = GAPDH (sense)
  source 1..18: mol_type = genomic DNA
acatcgctcagacccatg

SEQ ID NO: 4 - DNA, 22 bp, Homo sapiens
  misc_feature 1..22: note = GAPDH (antisense)
  source 1..22: mol_type = genomic DNA
tgtagttgaggtcaatgaaggg

SEQ ID NO: 5 - DNA, 21 bp, Homo sapiens
  misc_feature 1..21: note = DCN A (sense)
  source 1..21: mol_type = genomic DNA
aaatgcccaaaactcttcagg

SEQ ID NO: 6 - DNA, 21 bp, Homo sapiens
  misc_feature 1..21: note = DCN A (antisense)
  source 1..21: mol_type = genomic DNA
aagccccattttcaattcctg

SEQ ID NO: 7 - DNA, 20 bp, Homo sapiens
  misc_feature 1..20: note = Ki67 (sense)
  source 1..20: mol_type = genomic DNA
ctgcccagtggaagagttgt

SEQ ID NO: 8 - DNA, 20 bp, Homo sapiens
  misc_feature 1..20: note = Ki67 (antisense)
  source 1..20: mol_type = genomic DNA
cgaccccgctccttttgata

SEQ ID NO: 9 - DNA, 21 bp, Homo sapiens
  misc_feature 1..21: note = Alpha SMA (sense)
  source 1..21: mol_type = genomic DNA
tcctgtttgctgatccacatc

SEQ ID NO: 10 - DNA, 20 bp, Homo sapiens
  misc_feature 1..20: note = Alpha SMA (antisense)
  source 1..20: mol_type = genomic DNA
aatgcagaaggagatcacgg

(19) United States
(12) Patent Application Publication
(10) Pub. No.: US 2025/0049855 A1
(43) Pub. Date: Feb. 13, 2025

(54) ANTI-FIBROUS CELLS, MEDICAMENT COMPRISING THE CELLS, AND METHOD FOR OBTAINING THESE CELLS

(71) Applicant: B. Braun Avitum AG, Melsungen (DE)

(72) Inventor: Massimo Dominici, Ferrara (IT)

(21) Appl. No.: 18/720,003

(22) PCT Filed: Dec. 16, 2022
(86) PCT No.: PCT/EP2022/086318

§ 371 Date: Jun. 14, 2024

(30) Foreign Application Priority Data
Dec. 16, 2021 (IT) 102021000031517

(51) Int. Cl. A61K 35/28; C07K 14/47; C12N 5/0775; C12N 15/86

(52) U.S. Cl. CPC A61K 35/28; C07K 14/4725; C12N 5/0663; C12N 5/0668; C12N 15/86; C12N 2510/00; C12N 2740/10043; C12N 2740/15011; C12N 2840/20

(57) ABSTRACT

A method to produce modified mesenchymal cells, in particular for the treatment of renal fibrosis, includes modifying the cells with a viral vector, in such a way that the modified cells code for a protein which is Decorin. The modified mesenchymal cells are modified with a modifying agent that includes a viral vector associated with the mesenchymal cells so that the modified mesenchymal cells express Decorin. A medicament, in particular for the treatment of renal and lung fibrosis, includes the mesenchymal cells modified with the modifying agent and which express Decorin.

### CROSS-REFERENCE TO RELATED APPLICATIONS

This application is the United States national stage entry of International Application No. PCT/EP2022/086318, filed on Dec. 16, 2022, and claims priority to Italian Application No. 102021000031517, filed on Dec. 16, 2021. The contents of International Application No. PCT/EP2022/086318 and Italian Application No. 102021000031517 are incorporated by reference herein in their entireties.

### FIELD

The invention concerns modified cells, generally usable for preventing or treating the formation of fibrosis in lungs and kidney, a medicament comprising the modified cells, and a method to obtain the modified cells.

### BACKGROUND

It is known that there are many causes of fibrosis and malfunction of different tissues, such as lungs and kidney, that have in common the limited ability of the organ itself to regenerate after repeated and consistent lesions.

These lesions cause the activation and migration of fibroblasts, leading to the deposition of proteins with an extra-cellular matrix, mainly collagen, and the secretion of cytokines that mediate with the cells of the immune system in the process known as fibrosis (Mullins, L. et al; “Disease Models and mechanisms”; 2016.9.1419-1433).

TGF-β, also called TGF-Beta, represents one of the most important cytokines involved in the fibrotic process.

This cytokine causes the transformation of epithelial cells into fibroblastoid cells, through the process known as the “epithelium-mesenchymal transition”, which results in an increase in the number of fibroblasts and a significant deposition of collagen (Kalluri et al.; J Clin Inv. 2003).

Based on this state of the art, the use of a decoy receptor, able to recognize and interact specifically with TGF-β, thus preventing it from binding with target epithelial cells, could represent a possible strategy to contrast the fibrosis process (Lan H Y; International Journal of Biological Sciences 2011), in particular for renal and lung tissues (Border W A et al. Nature. 1992; https://www.ncbi.nlm.nih.gov/pmc/articles/PMC3812949/Yue X et al. Curr Enzym Inhib. 2010).

Mesenchymal cells (hereinafter also MCs for short) represent the ideal cellular vehicle to carry and release the TGF-β decoy receptor in the fibrotic kidney and lungs, since both tissues are reported to be linked for several pathophysiological events (https://journals.physiology.org/doi/full/10.1152/ajplung.00152.2021; Bollenbecker S et al. American Journal of Physiology. Lung Cellular and Molecular Physiology 2022; https://link.springer.com/article/10.1007/s40620-018-00563-1; Sorino C et al. Journal of Nephrology 2018).

MC have the ability to move and take root in the damaged site since they have been recruited from soluble molecules released into the bloodstream by damaged leukocytes and cells (Di Marino A M. Front Immunol. 2013).

Thanks to their presence in numerous tissues (including adipose tissue, bone marrow, umbilical cord), their easy isolation, the possibility of genetic manipulation and their ability not to induce an immunogenic response, they represent the ideal vector in bringing compounds with a therapeutic purpose, such as the TGF-β decoy receptor, in cell-based therapeutic approaches.

This state of the art has some disadvantages.

One disadvantage is that renal fibrosis is treated with invasive and disabling therapies such as dialysis.

It is known that dialysis helps the kidneys to filter blood from its waste products, but it is not a cure for those who use it.

In fact, it is a treatment that brings no cure and, depending on the severity of the condition of the dialysis patient, it must be performed weekly or daily.

Another disadvantage is that, in addition to being very painful for patients, dialysis is also psychologically disabling, since it leads to discomfort for the person and physical discomforts that generate hypotension, itching, insomnia, pain and sexual disorders.

Added to the above is the further disadvantage that dialysis is a therapy that can only be performed in hospital, obliging patients to move from their homes to be able to perform periodic treatments and this implies a further disadvantage when patients have difficulty in walking.

In fact, since it is a complex therapy, dialysis requires specific machinery and the assistance of healthcare personnel who are experienced in their use.

Another disadvantage is that, in addition to being the only treatment known to date for kidney fibrosis, dialysis is also a very expensive therapy that is charged to the health systems of the European Community for amounts comprised between 14 and 15 billion euros, including the costs of machinery, the costs of reagents, the cost of staff and hospital beds.

In addition, patients with severe kidney disease are subjected to transplants, which is not always possible and burdened by side effects and high social and health costs.

Finally, another disadvantage must also be underlined, namely that, in cases in which the state of kidney fibrosis is very advanced, it is not possible to use dialysis, since this would not bring any benefit to the functionality of the kidney.

Similarly, and with an impressive and yet uncurable impact in cardiopulmonary medicine, lung fibrosis is associated with a very relevant health and social impacts (https://openres.ersjournals.com/content/4/2/00045-2017: Hilberg O et al. ERJ Open Research 2018).

One of the pivotal drivers in fibrosis is represented by TGF-β, playing a role in acute/chronic kidney and lung diseases but also in cancer (https://www.sciencedirect.com/science/article/pii/S1359610105001103: Bierie B & Moses H L. Cytokine & Growth Factor Reviews 2006). For this reason, attempts to counteract TGF-β actions have been previously described.

US-A1-2019/125804 discloses a method for treating cancer in a subject by administering a human umbilical cord perivascular cells (HUCPVC) that have been genetically modified to increase the expression of an oligonucleotide, antibody or polypeptides, in particular Decorin, as TGF-β decoy binder (https://www.sciencedirect.com/science/article/pii/S0021925820621263: Ferdous Z et al. JBC 2007). A drawback of that solution relates to the modification of HUCPVC made by a recombinant adenovirus that induces a not stable modification, therefore Decorin production in the HUCPVC. Moreover, from an immunogenic point of view, undesirable collateral effects are possible due to the immunogenicity of the adenovirus (https://www.frontiersin.org/articles/10.3389/fimmu.2020.00909/full: Coughlan L. Frontiers in Immunology 2020).

In addition, US-A1-2019/117701 discloses mesenchymal stem cells potentially having a therapeutic effect against various conditions, including fibrosis. In particular. Those mesenchymal stem cells express at least one cell surface marker selected from the group consisting of CD201, CD46, CD56, CD147 and CD165. The mesenchymal stem cells that express such a marker are also positive for CD29, CD73, CD90, CD105 and CD166 and maintain an undifferentiated state. US-A1-2019/117701 describes mesenchymal stem cells spontaneously secreting Decorin, amongst a large variety of additional molecules. That document does not suggest any modification of the mesenchymal stem cells to produce (or to enhance) Decorin production in an enforced manner. Moreover, the main feature of the mesenchymal stem cells is that of expressing at least one cell surface marker selected from the group consisting of CD201, CD46, CD56, CD147 and CD165, not Decorin. Moreover, as for US-A1-2019/125804, the described cells are derived from umbilical cord, adipose or bone marrow without any minimal suggestion on the use of MC from ET. This is particularly different since, as visible in FIG. 2 of the present application, cells from ET do not spontaneously express Decorin.

### SUMMARY

The purpose of the invention is to overcome the disadvantages described above, perfecting modified MCs for the treatment of renal and lung fibrosis and a method to obtain the modified cells that allow to resolve the disadvantages as above, in particular that allow to definitively treat fibrosis, leading to a resolution of the pathology.

Another technical purpose of the invention is to perfect a new treatment for renal fibrosis in which the treatment is not painful and psychologically disabling for the patient.

A further technical purpose of the invention is to perfect a new treatment for lung fibrosis that associated with very significant levels of morbidity and mortality.

Yet another technical purpose of the invention is to be able to be administered as a drug, preventing patients from having to go to the hospital, since it is easily accessible even for people who have mobility issues and high levels of oxygen demand.

Another technical purpose of the invention is to significantly reduce the costs borne by healthcare systems, since it does not require using specific and expensive machinery to perform dialysis, expert personnel present 24 hours a day in the hospital, specific reagents and, not yet resolutive, costly anti-fibrotic drugs.

Another technical purpose of the invention is to allow for the treatment of patients suffering from particularly advanced states of renal fibrosis, no longer treatable with dialysis.

According to one aspect of the invention, mesenchymal cells modified with a modifying agent are provided for use in the treatment of fibrosis of the kidney or of the lung.

In accordance with embodiments, the mesenchymal cells originate from endometrial tissue.

This differs from prior art solution known for example from US-A1-2019/125804, where the cells were derived from umbilical cord perivascular tissue.

According to embodiments, the mesenchymal cells are living cells taken from an endometrial decidual necrotic material.

According to another aspect of the invention, a method is provided to produce MCs modified with a modifying agent.

According to another aspect of the invention, the modifying agent is selected from Decorin isoform A, Decorin isoform B, Decorin isoform C, Decorin isoform D, Decorin isoform E.

According to another aspect of the invention, a method is provided to produce MCs modified with a modifying agent and expressing the molecule called HLA-G, with an anti-fibrogenic and immunosuppressive effect.

According to another aspect of the invention, there is provided a medicament, in particular to prevent or slow down renal fibrosis.

According to another aspect of the invention, there is provided a medicament for use in preventing or slowing down kidney fibrosis.

Also mesenchymal cells modified with a stable modifying agent are disclosed wherein the modifying agent is a viral vector selected from a lentivirus and a retrovirus.

The invention allows to obtain the following advantages:
treat patients affected by renal fibrosis, achieving complete recovery, even in the case of a pathology in an advanced state;
allow for the treatment of patients without needing specific machinery, expert personnel, reagent materials, significantly reducing treatment costs;
prevent the need for patients to travel to/from home and hospital, and vice versa;
reduce kidney transplant procedures;
eliminate any discomfort and pain for the patients treated.

According to another aspect of the invention, there is provided a medicament, in particular to prevent or slow down lung fibrosis.

According to another aspect of the invention, there is provided a medicament for use in preventing or slowing down lung fibrosis.

The invention allows to obtain the additional following advantages:
treat patients affected by lung fibrosis, achieving complete recovery, even in the case of a pathology in an advanced stage;
allow for the treatment of patients without needing specific machinery, specific anti-fibrotic drugs, expert personnel/caregivers, reagent materials, significantly reducing treatment costs;
prevent the need for patients to travel to/from home and hospital, and vice versa;
eliminate any discomfort and oxygen dependance for the patients treated.

### BRIEF DESCRIPTION OF THE DRAWINGS

Other characteristics and advantages of the invention will become apparent from the detailed description of some preferred, but not exclusive, embodiments of MCs modified with a modifying agent, of a method to produce MCs modified with a modifying agent, of a medicament, in particular to prevent renal and lung fibrosis, all of which are shown by way of a non-limiting example in the attached drawings wherein:

FIG. 1 is a graphic representation of the viral vector used to modify the MCs with the gene coding for Decorin;

FIG. 2 is a graphic representation of the quantity of Decorin secreted in the culture medium by three different sources of MCs, two donors for each source, at three different times;

FIG. 3A is a representative cytofluorimeter analysis of a sample of control endometrial mesenchymal cells to evaluate the positivity to the Decorin signal sequence;

FIG. 3B is a representative cytofluorimeter analysis of a sample of infected ET-MCs to evaluate the positivity to the Decorin signal sequence. The genetically modified sample was positive at 89.5%;

FIG. 4 is a graphic representation of the quantity of m-RNA for Decorin, normalized on the basal expression in ET-MCs not over-expressing Decorin;

FIG. 5 is a graphic representation of the quantity of Decorin secreted in the culture medium for control cells against the cells modified to over-express Decorin, detected at three different times;

FIG. 6A is a representative cytofluorimeter analysis of a sample of MCs isolated from adipose tissue (AT-MCs) and used as control to evaluate the positivity to the Decorin signal sequence;

FIG. 6B is a representative cytofluorimeter analysis of a sample of control AT-MCs to evaluate the positivity to the Decorin signal sequence. The genetically modified sample is positive in 54.5% of the cells in culture;

FIG. 7 is a graphic representation of the quantity of m-RNA for Decorin, normalized on the basal expression in MCs not over-expressing the target protein;

FIG. 8 is a graphic representation of the quantity of Decorin secreted in the culture medium for control AT-MCs cells in the comparison with AT-MCs modified to over-express Decorin, at three different times;

FIG. 9 is a graphic representation of the number of fibroblasts from idiopathic pulmonary fibrosis (IPF)CC-7231 migrated in the presence/absence of the empty vector ET-MCs and ET-MCs expressing Decorin after 48 hours of culture, on the basis of an assay conducted on isolated cells from three donors;

FIG. 10 is a graphic representation of the expression of the Ki-67 gene as a marker of proliferation of fibroblasts in co-culture for 48 hours with empty vector ET-MCs and ET-MCs expressing Decorin, on the basis of an assay conducted on two donors;

FIG. 11 is a graphic representation of the expression of the aSMA gene, a marker of the pro-fibrotic activity of fibroblasts in co-culture for 48 hours with empty vector ET-MCs and ET-MCs expressing Decorin;

FIG. 12 is a graphic representation summarizing the analysis by means of scanning microscopy (SEM) of biomimetic 3D models of dermal and kidney fibrosis obtained by means of bioprinting technology;

FIG. 13 is the evaluation of the expression of the fibronectin protein in biomimetic 3D models of fibrosis obtained by means of bioprinting technology using fibroblasts from the dermis;

FIG. 14 is the evaluation of the expression of the fibronectin protein in biomimetic 3D models of fibrosis obtained by means of bioprinting technology using kidney fibroblasts; and

FIG. 15 is HLA-G expression in EDT-MC with or without Decorin transduction.

### DETAILED DESCRIPTION

In accordance with the present invention, a method to produce modified mesenchymal cells MCs provides to modify the MCs with a modifying agent, in particular a modifying agent comprising a viral vector that codes for a protein. This protein is Decorin and the modified MCs express this Decorin.

Advantageously, the step of modifying the MCs comprises infecting the MCs with the viral vector.

Decorin is selected from Decorin isoform A, or isoform B, or isoform C, or isoform D, or isoform E.

The viral vector is preferably selected from a lentivirus or a retrovirus, since it is linked with a stable Decorin production.

According to the invention, there are also provided MCs modified with a modifying agent which are obtainable with the above method. In particular, the modifying agent comprises a retroviral vector infecting the MCs, and the MCs infected with the retroviral vector are infected MCs expressing Decorin.

The Decorin is selected from Decorin isoform A, or isoform B, or isoform C, or isoform D or isoform E, more preferably it is Decorin isoform A.

The MCs can be selected from MCs of human origin or animal origin. The MCs can also be selected from autologous or allogeneic MCs. It is also possible to provide that the MCs are selected from MCs coming from adipose tissue, or from bone marrow, from endometrial tissue, from placental tissue, from peripheral blood, from umbilical cord blood, from amniotic fluid and/or derivatives. In a preferred form, the MCs come from endometrial tissue.

Advantageously, the Decorin expressed by the modified mesenchymal cells is Decorin expressed repetitively.

The modified MCs as above can be provided for use in the treatment of the fibrotic process of lung and kidney. In particular, the invention provides MCs modified with a viral vector selected from lentiviral or retroviral and expressing Decorin as indicated above, for use as a medicament for the treatment of fibrosis.

In particular, the present invention also provides a medicament comprising MCs modified with a modifying agent as indicated above, wherein the modifying agent comprises a viral vector selected from lentiviral or retroviral infecting the modified MCs comprising modified cells expressing Decorin.

An isoform of Decorin conventionally called Decorin isoform A was selected, and hereafter referred to with this name for convenience of description, which is also known with the acronym DCN A.

DCN A is the most characterized of the decoy receptor isoforms for TGF-β (Zhang L et al, Aging, 2021).

For the present invention, reference has been made preferably to DCN A, that is, the one most characterized from the biological point of view.

### 1—Materials and Methods.

### 1.1—Isolation and Culture of the MCs.

1.1.1—MCs from endometrial tissue (ET): blood from endometrial tissue (called menstrual blood) was collected from healthy volunteer donors (n=2, denominated ET-MCs donor #1 and ET-MCs donor #2) during the first days of the menstrual cycle.

Each donor was provided with a menstrual cup, (DivaCup, Diva International, San Francisco, CA, USA) to collect the blood, which was subsequently transferred to a saline solution (PBS, PAA Laboratories, Pasching, Austria) with 1% penicillin/streptomycin (10,000 U/mL Penicillin, 10 mg/mL Streptomycin 0.9% NaCl solution, PAALaboratories), 35 mg/mL fluconazole (Diflucan, Pfizer, New York, NY, USA) and heparin (500 U/mL, Sigma, St. Louis, MO, USA).

The samples were kept at 4° C. for 24-48 h after collection, until they reached the laboratory dedicated to the treatment.

The endometrial tissue, when present, was discarded, and the remaining blood was homogenized by means of 20 passages through a 19G needle and using a 10 ml syringe.

The cell suspension was cultured as described below.

1.1.2—MCs from adipose tissue (AT): AT-MCs were obtained from lipoaspirate samples (n=2 denominated AT-MCs donor #1 and AT-MCs donor #2) collected from subcutaneous tissue from healthy donors by means of liposuction.

The samples were used having got the patient's informed consent and following approval of the study by the Local Ethics Committee (Azienda Universitario-Ospedaliera, Policlinico di Modena); within the frame of the project entitled “Development of novel anti-tumor therapies based on the use of mesenchymal/stromal progenitors isolated from adipose tissue Policlinico Protocol”, protocol nr. 0004827/20.

The lipoaspirate (1 cc) was treated to obtain AT-MCs precursors by means of extensive washing with equivalent volumes of saline solution (PBS Code: 14190-094. GIBCO, Thermo Scientific, Waltham, MA, USA) and subsequently digested at 37° C. for 30 minutes with 0.075% collagenase.

The enzymatic activity was subsequently neutralized with DMEM (Code: 41966-029. GIBCO, Thermo Scientific, Waltham, MA, USA), with 10% heat inactivated FBS (Code: SH30070.03. HyClone Laboratories, Inc, Logan, Utah, USA), and centrifuged at 1200 g for 10 minutes to obtain a cell pellet.

The pellet was then resuspended in 160 mM NH4Cl (Code: A9434, Sigma Aldrich Inc, USA) and incubated at ambient temperature for 10 minutes to lysate the contaminating erythrocytes.

The cells obtained were then collected by centrifugation and then filtered through a 100 μm filter to remove cell debris and incubated overnight at 37° C./5% CO2 in control medium (DMEM, 10% FBS).

Following this incubation, the plates were washed several times with PBS (Code: 14190-094. GIBCO, Thermo Scientific, Waltham, MA, USA) to remove residual non-adherent erythrocytes.

1.1.3—MCs from bone marrow (BM): BM samples were collected from the posterior iliac crest.

The samples (denominated BM-MCs donor #1 and BM-MCs donor #2) were suctioned with a 10 mL Luer-lock syringe containing 0.5-1 mL Na citrate (38 mg/mL) (Code: PHR1416, Sigma Aldrich Inc, USA), and processed as follows. The BM was diluted 1:1 (v: v) with Ca 2/Mg2-free sterile PBS (PBS Code: 14190-094. GIBCO, Thermo Scientific, Waltham, MA, USA) and made to pass 20 times through a 10-mL sterile syringe (Becton Dickinson Plastipak, Drogheda, Ireland) with a 19-G needle. The cell suspension was cultured as described below. The samples were used having got the patient's informed consent and following approval of the study by the Local Ethics Committee (Azienda Universitario-Ospedaliera, Policlinico di Modena, within the frame of the study entitled “Cellular therapies for cancer”, protocol code nr. 335/CE).

### 1.2—Cell Culture Method

The AT-MCs and ET-MCs were cultured in αMEM (Code: 22561-021. GIBCO, Thermo Scientific, Waltham, MA, USA) with 2.5% PLP (Human Platelet Lysate. Code: BC0190030 Macopharma Italy SRL, Milan, Italy), 2 mM L-Glutamine (Code: ECB3000D. Euroclone SpA, Italy), 1 International Unit (IU)/mL heparin (Code: H3149. Sigma Aldrich Inc, USA), and 10 μg/mL ciprofloxacin (Code: A15571/AIT. Fresenius Kabi Italia Srl, Verona, Italy).

The BM-MCs were cultured in αMEM (Code: 22561-021. GIBCO, Thermo Scientific, Waltham, MA, USA) with 8% PLP (Human Platelet Lysate. Code: BC0190030 Macopharma Italy SRL, Milan, Italy), 2 mM L-Glutamine (Code: ECB3000D. Euroclone SpA, Italy), 1 IU/mL heparin (Code: H3149. Sigma Aldrich Inc, USA), and 10 μg/mL ciprofloxacin (Code: A15571/AIT. Fresenius Kabi Italia Srl, Verona, Italy).

Human embryonic kidney cells 293T, were cultured in DMEM (Code: 41966-029. GIBCO, Thermo Scientific, Waltham, MA, USA) with 10% FBS heat-inactivated defined serum (Code: SH30070.03. HyClone Laboratories, Inc, Logan, Utah, USA), 2 mM L-Glutamine (Code: ECB3000D. Euroclone SpA, Italy) and 1% penicillin/streptomycin (pen/strep, Code: MS00581009. Carlo Erba Reagents Srl, Cornaredo, Italy).

Human fibroblasts isolated from non-pathological kidney were acquired (Code: H6016, Cell Biologics Inc., USA) as cryopreserved cells. After thawing, the cells were cultured in Complete Fibroblast Medium (M2267-Kit, Cell Biologics Inc., USA) which, in addition to the base culture medium, is constituted by the following supplements: FGF 0.5 mL of FGF; 0.5 mL of hydrocortisone; 5 mL of L-Glutamine; 5 mL of antifungal antibiotic solution; 50 mL of FBS. The volumes are correlated to 500 mL of culture medium. The cells are cultured in pre-treated flasks for 20-30 minutes at 37° C. with a gelatin-based conditioning solution (Code: 6950, Cell Biologics Inc, USA) capable of increasing the adhesive capacity of the cells.

Human fibroblasts isolated from the dermis were acquired from ATCC as cryopreserved cells. Once thawed, the cells kept under culture with DMEM (Code: 41966-029. GIBCO, Thermo Scientific, Waltham, MA, USA) with 10% FBS (Code: SH30070.03. HyClone Laboratories, Inc, Logan, Utah, USA), 2 mM L-Glutamine (Code: ECB3000D. Euroclone SpA, Italy) and 1% penicillin/streptomycin (pen/strep, Code: MS00581009. Carlo Erba Reagents Srl, Cornaredo, Italy).

Human lung fibroblasts from idiopathic pulmonary fibrosis were acquired in passage 2 in frozen vials (Cat: CC-7231; Lonza Group Ltd.), hereafter referred to as CC7231.

Hypertrophic fibroblasts, from idiopathic pulmonary fibrosis, were selected as target cells for the study since they are commercially available, unlike kidney hypertrophic fibroblasts, and since they share with the latter the biological mechanism at the basis of the fibrotic process and the same target for Decorin (TGFβ; Ong C H et al, European Journal of Pharmacology, 2021).

Upon receipt, they were thawed in DMEM (Code: 41966-029. GIBCO, Thermo Scientific, Waltham, MA, USA) with 10% heat-inactivated FBS (Code: SH30070.03. HyClone Laboratories, Inc, Logan, Utah, USA), 2 mM L-Glutamine (Code: ECB3000D. Euroclone SpA, Italy) and 1% penicillin/streptomycin (pen/strep, Code: MS00581009. Carlo Erba Reagents Srl, Cornaredo, Italy) and sown at a density of 2500/cm2 and cultured in the following medium: CLONETICS™ FGM™-2 BULLETKIT™ (Cat: CC-3132; Lonza Group Ltd.).

### 1.3—Production of the Virus and Infection of the MCs.

293T cells were transfected using the transfection reagent JetPEI DNA (Code 101-40N. Polyplus transfection, Illkirch, France) using a combination of an expression vector and helper plasmids: the viral vector (manufactured by OriGene Technologies Inc.).

1 mL of retroviral supernatant produced by the transfected 293T to which 6 μg/mL of polybrene (Code TR1003. Sigma Aldrich Inc. USA) was added, was used to transduce 45000/cm2 of ET-MCs and AT-MCs at early passages (p2/p4). Infections were repeated twice and followed by cell expansion and selection in Puromycin 2 μg/mL.

The viral supernatants were used fresh and/or frozen at −80° C.

### 1.4—Cytofluorimeter Analysis.

Intracellular staining for MYK on wild type and transduced ET-MCs and AT-MCs was performed with the Becton Dickinson Cytofix/Cytoperm Kit (Code: 554714. BD, Franklin Lakes, NJ, USA).

To evaluate MYK expression, the MCs were labeled with the primary antibody mouse anti-human MYK (Code: TA150121, OriGene Technologies Inc.) and then with the secondary antibody APC goat anti-mouse Ig (APC Goat AntiMouse Ig polyclonal multiple adsorption; Code: 550826. BD, Franklin Lakes, NJ, USA).

To evaluate HLA-G expression, ET-MCs were marked with the primary anti-HLA-G FITC-conjugated monoclonal antibody (MoAb) (87G, Exbio, Praha, Czech Republic) APC goat anti-mouse Ig (APC Goat AntiMouse Ig polyclonal multiple adsorption; Cod: 550826. BD, Franklin Lakes, NJ, USA).

The data were collected using the FACS Aria III (BD) flow cytometer and analyzed using the FACS Diva software (BD).

### 2—Molecular Biology.

Total RNA was isolated using TRIZOL® (Code: 15596026. Invitrogen, Carlsbad, MN, USA) following the instructions for use.

The cDNA was subsequently synthesized from 2 μg total of RNA using the kit RevertAid H minus first-strand cDNA synthesis (Code: K1622. Fermentas ThermoFisher, Waltham, MA, USA).

The cDNA was quantified using a spectrophotometer. (Beckman Coulter DU® 730, Pasadena, CA, USA).

Quantitative real-time PCR (qRT-PCR) was performed using the Applied Biosystems STEPONE™ Real-Time PCR System and the reagent Fast SYBR® Green Master Mix.

The qRT-PCR reaction (10 μl) consists of 50 ng cDNA, Fast SYBR Green Master Mix (Code: 4385612. Applied Biosystems, Foster City, CA, USA), and 300 nM of the forward and reverse primers.

The sequences of the respective primers are described in Table 1.

The relative expression levels of the target genes were calculated by means of the 2-ΔΔCt method using human β-actin and GAPDH genes as control genes.

TABLE 1

| | | Amplified |
|---|---|---|
| | | Length |
| Gene | Primer sequence | (bp) |
| β-actin | 5′-ACCTTCTACAATGAGCTGCG-3′ | 148 |
| | (sense) | |
| | 5′-CCTGGATAGCAACGTACATGG-3′ | |
| | (antisense) | |
| GAPDH | 5′-ACATCGCTCAGACCCATG-3′ | 148 |
| | (sense) | |
| | 5′-TGTAGTTGAGGTCAATGAAGGG-3′ | |
| | (antisense) | |
| DCN A | 5′-AAATGCCCAAAACTCTTCAGG-3′ | 146 |
| | (sense) | |
| | 5′-AAGCCCCATTTTCAATTCCTG-3′ | |
| | (antisense) | |
| Ki67 | 5′-CTGCCCAGTGGAAGAGTTGT-3′ | |
| | (sense) | |
| | 5′-CGACCCCGCTCCTTTTGATA-3′ | |
| | (antisense) | |
| Alpha | 5′-TCCTGTTTGCTGATCCACATC-3′ | |
| SMA | (sense) | |
| | 5′-AATGCAGAAGGAGATCACGG-3′ | |
| | (antisense) | |

### 2.1—ELISA Assay.

The levels of DCN A in the ET-MCs and AT-MCs samples were measured using the Duo Set Elisa kit (DY143. R&D Systems, 614 Mckinley Place NE, Minneapolis, MN, USA) following the instructions for use. This assay is based on the enzyme linked immunosorbent assay (ELISA) technique.

### 2.2—Migration Assays.

On day 1, the ET-MCs were seeded in plates of 24 wells at a concentration of 10000/cm2 (3 wells for each condition) in their culture medium.

The CC-7231s at a density of 10000/cm2 (3 wells for each condition) were seeded on Transwell Permeable Support (Code: 3422, Costar Corning Incorporated, USA) in DMEM (Code: 41966-029. GIBCO, Thermo Scientific, Waltham, MA, USA) with 2.5% of NUSERUM™ IV culture supplement (Code: 355104, BD Biosciences) inactivated with heat, 2 mM L-Glutamine (Code: ECB3000D. Euroclone SpA, Italy) and 1% penicillin/streptomycin (pen/strep, Code: MS00581009. Carlo Erba Reagents Srl, Cornaredo, Italy).

On day 4, the supports were removed, washed with DPBS 1× (Code: 14190-094, Gibco) and subsequently fixed with cold methanol. After fixation, they were further washed with distilled water, the layer of non-migrated cells was removed with a buffer and the remaining ones were stained with Crystal Violet 0.4% (Code: C0075 Sigma Aldrich), and after further washing in distilled water they were made to dry before viewing under a microscope and manual counting.

### 2.3—Proliferation Assay.

The CC-7231s at a density of 10000/cm2 (6 wells for each condition) were seeded in plates with 24 wells in DMEM (Code: 41966-029. GIBCO, Thermo Scientific, Waltham, MA, USA) with 2.5% of NUSERUM™ IV culture supplement (Code: 355104, BD Biosciences) inactivated with heat, 2 mM L-Glutamine (Code: ECB3000D. Euroclone SpA, Italy) and 1% penicillin/streptomycin (pen/strep, Code: MS00581009. Carlo Erba Reagents Srl, Cornaredo, Italy).

A co-culture with ET-MCs and fibroblasts from idiopathic fibrosis in a ratio of 1:1 was set up. In detail, the ET-MCs were sown in the bottom of the MW wells and the fibroblasts in a transwell housed above the ET-MCs culture, having a porosity of 3 μm capable of allowing the exchange of the medium and of the solutes contained therein. The co-culture thus obtained was protracted for 48 hours and subsequently the cells were lysed, RNA extracted with trizol (Code: 15596026. Invitrogen, Carlsbad, MN, USA) following the instructions for use.

The cDNA was subsequently synthesized from 2 μg total of RNA using the kit RevertAid H minus first-strand cDNA synthesis (Code: K1622. Fermentas ThermoFisher, Waltham, MA, USA).

The cDNA was quantified using a spectrophotometer (Beckman Coulter DU® 730, Pasadena, CA, USA).

Quantitative real-time PCR (qRT-PCR) was performed using the Applied Biosystems STEPONE™ Real-Time PCR System and the reagent Fast SYBR® Green Master Mix.

The qRT-PCR reaction (10 μl) consists of 50 ng cDNA, Fast SYBR Green Master Mix (Code: 4385612. Applied Biosystems, Foster City, CA, USA), and 300 nM of the forward and reverse primers of the Ki-67 gene.

3—Assessment assay of the expression of aSMA

The CC-7231s at a density of 10000/cm2 (6 wells for each condition) were seeded in plates with 24 wells in DMEM (Code: 41966-029. GIBCO, Thermo Scientific, Waltham, MA, USA) with 2.5% of NUSERUM™ IV culture supplement (Code: 355104, BD Biosciences) inactivated with heat, 2 mM L-Glutamine (Code: ECB3000D. Euroclone SpA, Italy) and 1% penicillin/streptomycin (pen/strep, Code: MS00581009. Carlo Erba Reagents Srl, Cornaredo, Italy).

A co-culture with ET-MCs and fibroblasts from idiopathic fibrosis in a ratio of 1:1 was set up. In detail, the ET-MCs were sown in the bottom of the MW wells and the fibroblasts in a transwell housed above the ET-MCs culture, having a porosity of 3 μm capable of allowing the exchange of the medium and of the solutes contained therein. After 24 and 48 hours of culture, the cells were lysed and RNA was extracted with TRIZOL.

The cDNA was subsequently synthesized from 2 μg total of RNA using the kit RevertAid H minus first-strand cDNA synthesis (Code: K1622. Fermentas ThermoFisher, Waltham, MA, USA).

The cDNA was quantified using a spectrophotometer. (Beckman Coulter DU® 730, Pasadena, CA, USA).

Quantitative real-time PCR (qRT-PCR) was performed using the Applied Biosystems STEPONE™ Real-Time PCR System and the reagent Fast SYBR® Green Master Mix.

The qRT-PCR reaction (10 μl) consists of 50 ng cDNA, Fast SYBR Green Master Mix (Code: 4385612. Applied Biosystems, Foster City, CA, USA), and 300 nM of the forward and reverse primers of the aSMA gene.

### 4—SEM Microscopy Analysis of Biomimetic 3D Models of Fibrosis by Means of Bioprinting Technology.

The biomimetic models were obtained by means of bioprinting and are based on the growth of fibroblasts isolated from the dermis or kidney in a three-dimensional matrix of natural origin. The analysis was carried out with SEM (TM4000 Plus II, Tabletop Microscope, Hitachi Technologies Corporation, Japan). The interaction between an electron beam and the atoms of the sample under examination allow to generate images with very high magnifications, and to analyze changes in the microarchitecture of the tissue. The micrographs were obtained with an acceleration of the electron beam of 10 kV, in medium vacuum conditions, and through the use of a secondary electrons detector at 1000× magnification.

### 5—Immunofluorescence (IF) Reaction for Fibronectin

The biomimetic models embedded in paraffin were microtome processed and cut into sections with a thickness of 4 micron. After drying at 37 degrees Celsius, the sections were de-paraffinized and rehydrated through a graded passage of alcohols at increasing concentrations. Following unmasking with citric acid (Code: 403727, Carlo Erba Reagents spa, Arese, Milan), the sections were incubated with anti-fibronectin primary antibody (Code: ab2413, Abcam) at a concentration of 1:100 for 1 hour at ambient temperature and with Donkey anti-rabbit secondary antibody IgG-h+I Dylight 594 conjugated anti rabbit (Code: A120-108D4, Bethyl) at a concentration of 1:700 for 1 hour at ambient temperature. The nuclei were stained for 5 minutes with the dye DAPI (Code: 10236276001, Roche) at a concentration of 1:200 for 5 minutes at ambient temperature. The sections were then mounted in buffered glycerin. The analysis was carried out with an AxioZoom V16 microscope (Zeiss) with 1× objective (Plan NeoFluar Z 1×/0.25 FWD 53.1 mm, Zeiss) and 180× digital magnification.

The quantification of the signal was obtained through the ImageAnalysis plugin of the ZEN Pro Zeiss software.

The result of the analysis expressed in μm2 was reported as a percentage, considering the negative control as 100% of the expression.

### 6—Results.

The gene for DCN A was cloned in a bicistronic viral vector in which the gene for Puromycin was also inserted (OriGene Technologies Inc. NM_001920).

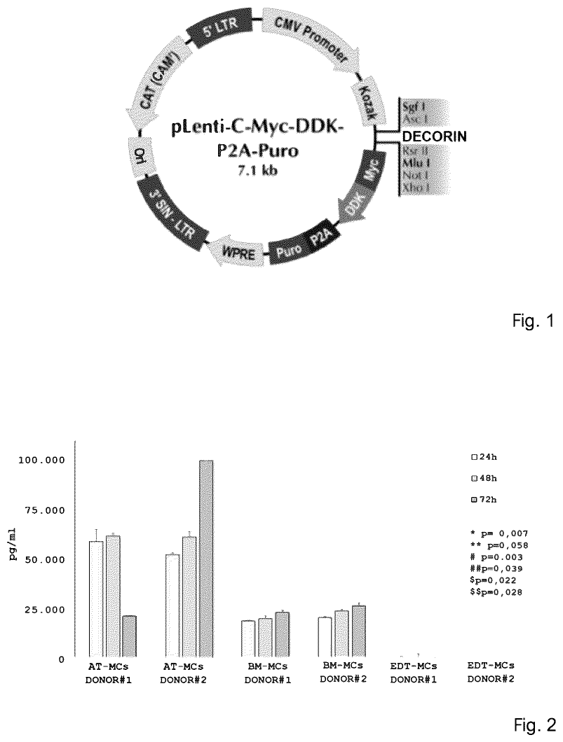

The viral vector (in this case preferably, but not exclusively, of the lentiviral type), represented in FIG. 1, with the gene inside was amplified in bacteria capable of producing large quantities of the DNA of interest and the empty vector was used as a negative control.

The bacterial product was purified and subsequently sequenced to characterize the gene sequence inserted in the lentiviral vector. The sequence analysis confirmed that no mutations occurred during the amplification step.

To identify the correct source of MCs by means of which to convey the therapeutic target, we isolated MCs from adipose tissue (AT), bone marrow (BM) and from endometrial tissue (ET), of which two donors for each source were isolated.

After isolation, the quantity of Decorin isoform A physiologically produced by AT-MCs, BM-MCs, ET-MCs wild type cells was analyzed in early passages (P2) at different times (24 h, 48 h, 72 h) in the supernatants collected from two donors for each cell type, as shown in FIG. 2.

The AT-MCs have been shown to secrete the greatest quantity of DCN A, ranging comprised between 22085 and 99774 μg/mL.

In contrast, the ET-MCs secrete a low quantity of DCN A, ranging from 206 to 688 μg/mL, more than 30 times less than AT-MCs.

BM-MCs have been shown to secrete an intermediate quantity, between the previously mentioned sources of DCN A, varying between values of 19425 and 24863 μg/mL.

These data proved to be unpredictable for the person of skill, for the reason that until the present invention the secretion of DCN A in MCs was never clearly stimulated, nor was this ever suggested to the person of skill, either by the literature or by previous documents, directly or indirectly.

### 7—Endometrial Mesenchymal Cells (ET-MCs).

Starting from the known basal secretion, the lowest among the sources analyzed so far, of DCN A by the MCs and by the sequenced bicistronic vector, three donors of ET-MCs were infected.

To purify the ET-MCs after infection and to obtain a population completely pure for DCN A, the cells were selected with Puromycin for 96 h.

The dose for selecting cells was optimized in a range that varies from 0.5 μg/mL to 5 μg/mL of antibiotic.

To verify and quantify the efficiency of infection with the DCN A vector, the ET-MCs were analyzed for their positivity to MYK, a signal sequence inserted in the plasmid and correlated to the expression of the gene for DCN A.

This signal sequence allows to evaluate the quantity of protein induced in the modified cells, diverging it from the endogenous one.

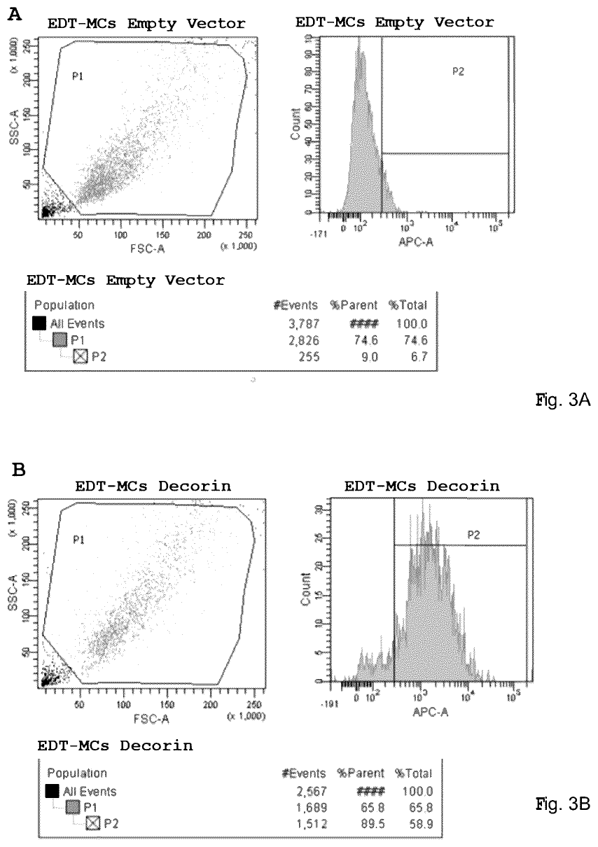

The donor of ET-MCs induced to express DCN A shows a positivity of 89.5% compared with the control expressing an empty vector, thus indicating a good infection efficiency (FIGS. 3A, 3B).

To quantify the expression of the gene for DCN A in the infected ET-MCs, their m-RNA was collected, back-transcribed into cDNA and analyzed by means of RT-PCR.

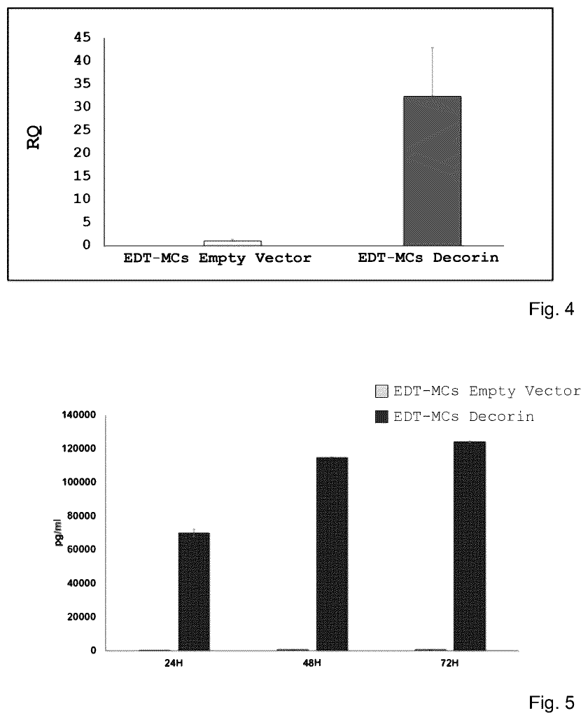

FIG. 4 shows how the gene for DCN A is over-expressed in the samples after infection and the corresponding expression (RQ.ET-MCs) is 32.38±10.45 times higher than in the empty vector control.

The ELISA assay was performed to compare the quantity of protein released into the medium by the empty vector ET-MCs or the ET-MCs infected for DCN A.

The quantity of isoform A released into the culture medium, as indicated in FIG. 5, increases until a difference between infected ET-MCs and empty vector is reached that is 230 times greater.

Furthermore, the simultaneous production of HLA-G by ET-MC cells was evaluated by means of FACS analysis. From the analysis carried out it was deduced that the ET-MCs cells modified for Decorin express levels of HLA-G for a proportion that is bigger with respect to ET-MC empty vector.

8—Mesenchymal Cells from Adipose Tissue (AT-MCs).

Starting from the known basal secretion, the highest among the previously analyzed sources, of DCN A by the MCs and by the sequenced bicistronic vector, two donors of AT-MCs were infected.

To purify the AT-MCs after infection and to obtain a population completely pure for DCN A, the cells were selected with Puromycin for 96 h.

The dose for selecting cells was optimized in a range that varies from 0.5 μg/mL to 5 μg/mL of antibiotic.

To verify and quantify the efficiency of infection with the DCN A vector, the AT-MCs were analyzed for their positivity to MYK, a signal sequence inserted in the plasmid and correlated to the expression of the gene for DCN A.

This signal sequence allows to evaluate the quantity of protein induced in the modified cells, diverging it from the endogenous one.

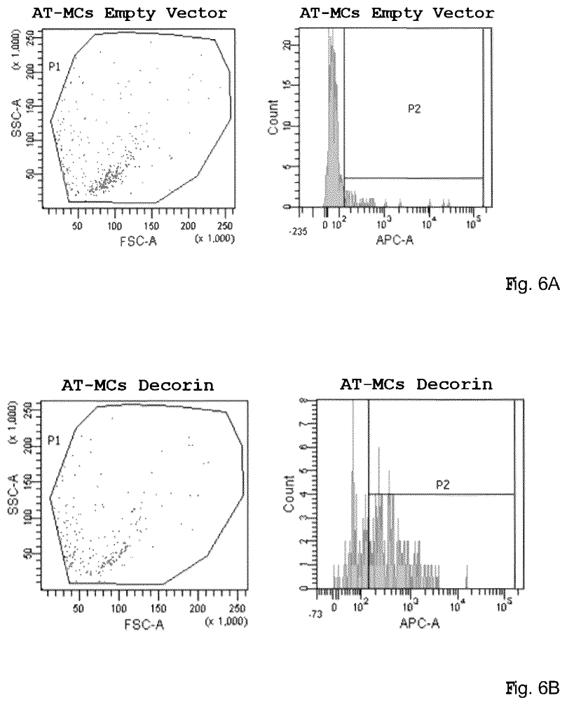

The donor of AT-MCs induced to express DCN A shows a positivity of 54.5% compared with the control expressing an empty vector, thus indicating a good infection efficiency (FIGS. 6A-6B).

To quantify the expression of the gene for Decorin isoform A in the transduced AT-MCs, their m-RNA was collected, back-transcribed into cDNA and analyzed by means of RT-PCR.

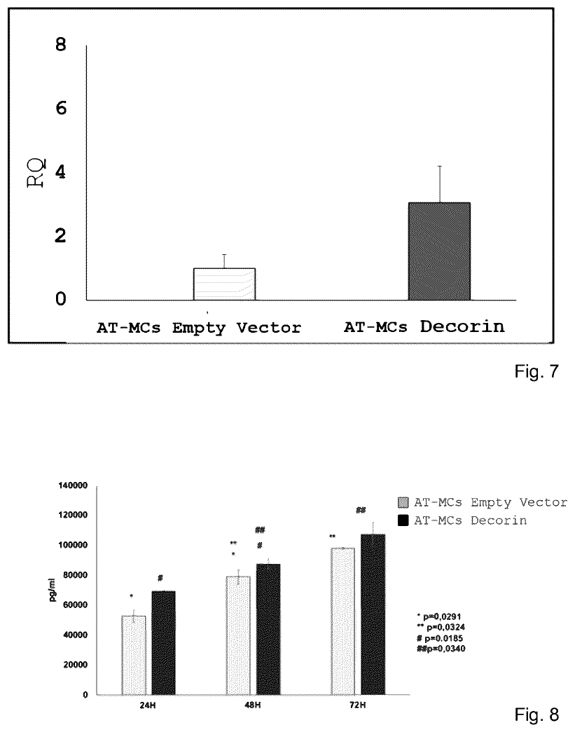

FIG. 7 shows how the gene for DCN A is over-expressed in samples after infection and the corresponding expression (RQ.AT-MCs) is 3.05±1.14 times higher than in the empty vector control).

The ELISA assay was performed to compare the quantity of protein released into the medium by the empty vector AT-MCs or by the AT-MCs infected for DCN A.

The quantity of such Decorin isoform A released into the culture medium, as indicated in FIG. 8, increases in a statistically significant way even though it does not reach the absolute quantity that is reached after modification of the ET-MCs mentioned above. This phenomenon could be caused by the already high basal secretion of the AT-MCs (with respect to the basal secretion of isolated MCs from other sources) which interferes with the production and/or stability of the protein induced by the vector of interest.

Having verified the difference in absolute quantity of secreted protein, between ET-MCs and AT-MCs modified for the aforementioned DCN A, and having ascertained that the ET-MCs are able to produce a greater quantity of DCN A, value expressed in pg/mL, we proceeded to evaluate the efficacy of DCN A produced by the ET-MCs on target cells, that is, on hypertrophic fibroblasts.

### 9—Functional Studies.

Hypertrophic fibroblasts, from idiopathic pulmonary fibrosis, were selected as target cells for the study, since they are commercially available, unlike kidney hypertrophic fibroblasts, and since they share with the latter the biological mechanism underlying the fibrotic process and the same target for the DCN A, that is, TGFβ (Ong C H et al, European Journal of Pharmacology, 2021).

Following expansion, the hypertrophic fibroblasts were analyzed for their ability to migrate and proliferate in the presence of ET-MCs modified to express DCN A or the empty vector.

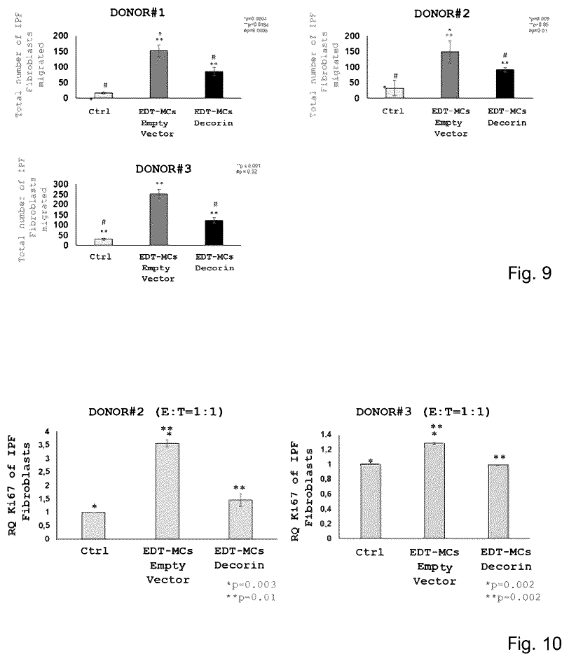

The migration study carried out by sowing the ET-MCs in plates and the fibroblasts above them, in a grid developed for the above studies, after a period of 48 hours shows how the CC-7231s cultured without ET-MCs have almost zero ability to migrate. When cultured with empty vector ET-MCs they increase their ability to migrate, by means of factors released in the medium, while when cultured with ET-MCs expressing DCN A, the ability to migrate is halved in all the donors examined. One hypothesis could be that the fibroblasts are attracted toward the ET-MCs, thanks to the release of the latter of chemo-attractive factors including the activation by TGFβ, which is one of the major activators of the migration of fibroblasts in a fibrotic process (Frangogiannis N G et al. J Exp Med. 2020). However, this phenomenon is slowed down in the presence of ET-MCs releasing DCN A, because the latter, by binding the TGFβ, can in turn prevent its binding with its receptor, decreasing the number of migrated fibroblasts, as shown in FIG. 9.

The study on the proliferation of hypertrophic fibroblasts (CC-7231) was performed by setting up the co-culture with empty vector ET-MCs and ET-MCs expressing Decorin, in the ratio of 1:1.

FIG. 10 shows the expression levels of the Ki-67 gene normalized on the culture of the fibroblasts, as indicated as control. The data showed that, if compared with the control represented by the culture medium alone, a statistically significant increase of the KI67 gene in co-culture with empty vector ET-MCs is observed, indicating a proliferative stimulus by factors released by the MCs. In co-culture with ET-MCs modified with Decorin, this proliferative stimulus was considerably reduced. This biological response may be caused by the decrease of TGFβ available in the specific culture medium used for maintaining the cell cultures, due to the lack of binding with its receptor thanks to the previous binding with the Decorin secreted by the modified ET-MCs. The result obtained suggests the role of Decorin in reducing proliferation and migration.

Finally, the expression of the aSMA gene was evaluated, a marker par excellence of the activation by fibroblasts in myofibroblasts, a typical phenotype of fibrosis, where cells with contractile capacity secrete extracellular matrix, worsening the pathological picture (Kuhn and McDonald, Am J Pathol 1991; Flaherty et al. Am. J. Respir. Crit. Care Med. 2003; White et al. J. Pathol. 2003; Hinz, Proc. Am. Thorac. Soc 2012).

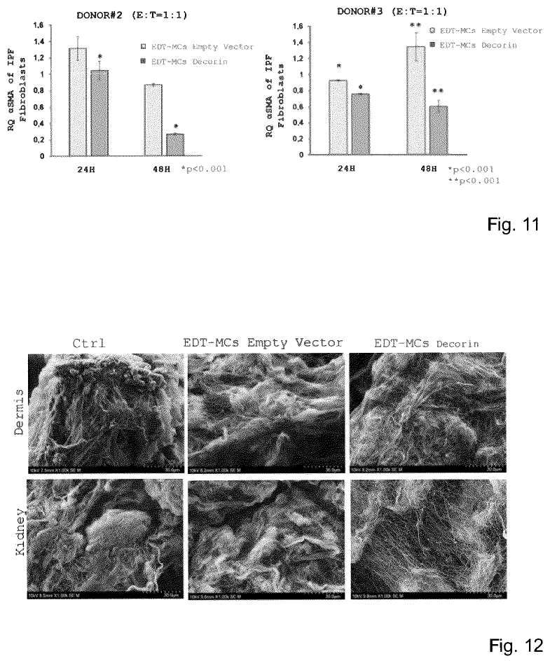

The result relating to the expression of the aSMA gene shown in FIG. 11 following co-culture in the ratio of 1:1 between idiopathic fibroblasts CC-7231 and empty vector ET-MCs and ET-MCs expressing Decorin highlights the role of the modified MCs. At both 24 hours as well as 48 hours of co-culture, a statistically significant reduction in the expression of aSMA is observed in fibroblasts in co-culture with ET-MCs expressing Decorin.

This result demonstrates a reduction in the activation state of hypertrophic fibroblasts due to the role of Decorin.

Having collected evidence regarding the role of Decorin in commercial fibroblasts isolated from idiopathic pulmonary fibrosis, we wanted to evaluate the effect on biomimetic matrices of the healthy and fibrotic dermis and the healthy and fibrotic renal stromal tissue.

Through printing technology, fibroblasts (isolated both from the dermis and also the kidney) were immersed in a three-dimensional matrix capable of mimicking the fibrotic pathology (FIG. 12). The untreated models (CNTRL) show a dense and compact surface with encapsulation of collagen fibrils by abundant extracellular matrix (left column). The supernatant obtained from ET-MCs with empty vector gives the model a solid surface characterized by partial remodeling of the collagen fibrils and poor deposition of extracellular matrix. On the other hand, the supernatant obtained from ET-MCs expressing Decorin significantly reduces the density of the printed tissue without an extra-cellular matrix with fibrils that appear thinner and more fragmented.

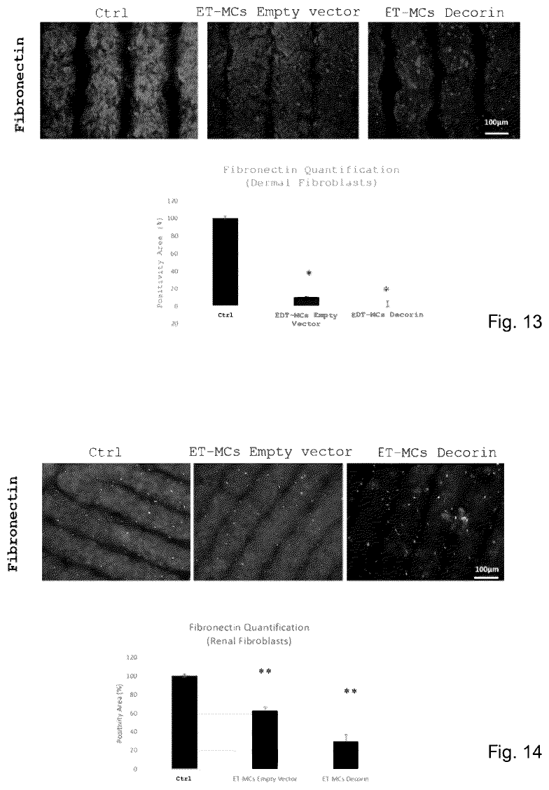

FIG. 13 shows the immunofluorescence detection of the fibronectin protein in the three-dimensional model obtained with fibroblasts from the dermis, as a further indicator of fibroses (O'Connell et al. Fibronectin: Current Concepts in Structure, Function and Pathology 2012).

The intensity and localization of the signal linked to the presence of fibronectin are particularly intense in the CNTRL case. On the contrary, the protein is scarcely present in cases stimulated with the supernatants of the MCs.

In particular, the lowest expression levels were observed following stimulation of the model with the supernatant of ET-MCs over-expressing Decorin.

The quantification of the staining confirms a statistically significant reduction of the fibronectin expression levels in the cases treated with supernatant obtained from ET-MCs and ET-MCs expressing Decorin, in a statistically significant way compared to the control case (p value <0.05). The biomimetic models treated with supernatant collected from ET-MCs expressing Decorin show significantly lower levels of expression not only compared to the control case but also in relation to ET-MCs.

The same analysis was carried out on three-dimensional models obtained with kidney fibroblasts.

In FIG. 14, the fluorescence (red) indicates the presence of the fibronectin protein in the model.

The intensity and localization of the signal linked to the presence of fibronectin are particularly intense in the CNTRL case. On the contrary, the protein is scarcely present in the cases treated with the supernatant of ET-MCs with empty vector and ET-MCs over-expressing Decorin. The quantification of the staining by means of Image Analysis plugin (ZEN PRO, Zeiss) confirms a statistically significant reduction of fibronectin expression levels in cases treated with supernatants of ET-MCs with empty vector and with ET-MCs over-expressing Decorin (p-value <0.05). In detail, the case treated with the supernatant of ET-MCs over-expressing Decorin shows significantly lower expression levels not only compared to the control case but also in relation to the case treated with supernatant of ET-MCs with empty vector.

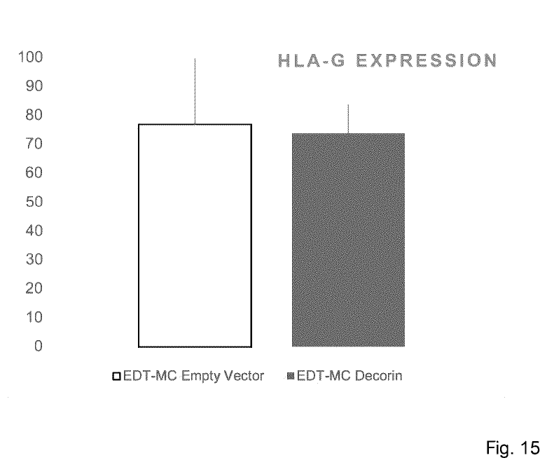

In FIG. 15, HLA-G was tested on both EDT-MC empty vector and EDT-MC modified to express Decorin. Test was performed by FACS using either FITC or APC conjugated anti-HLA-G monoclonal antibodies. Two different MC donors where considered. As visible, all samples express HLA-G without differences (p>0.05) related to Decorin gene modification.

### 10—Conclusions.

According to the invention, the endogenous secretion of DCN A by MCs coming from different sources was investigated.

Each source has shown to secrete a different level of protein secretion and among these ET-MCs have been selected as weakly secreting and AT-MCs as cells highly secreting the identified decoy receptor, to better understand the effect of the gene modification on the cells themselves.

The method of infection with the viral vector containing DCN A and the gene for Puromycin has been optimized leading to an efficient infection.

The MCs, after infection, did not show signs of suffering, morphology change and/or changes in the expression of typical markers of MCs.

The protocols for gene quantification by means of Real Time-PCR and for the quantification of the signal sequence by means of cytofluorimeter analysis were optimized, demonstrating an increase in the gene for DCN A.

The secretion of the protein was proven by means of ELISA assay, indicating an increase in the release in MCs induced to produce DCN A, confirming the data obtained by means of molecular analysis, which show a greater expression of the gene for DCN A, and data obtained by means of cytofluorimeter analysis in which the MCs induced to express DCN A show an increase in positivity compared to MCs with empty vector.

Furthermore, the comparison carried out between different sources of MCs led to select as the best source the ET-MCs that have demonstrated having a greater positivity to the MYC signal sequence by means of cytofluorimeter analysis, a greater increase in the expression of mRNA, which results in turn in the increase in the quantity of protein secreted. For this reason, ET-MCs represent the best carrier for the purpose of the present invention which is expressed in a cellular and gene therapy approach that can be performed with the invention.

To verify the functionality of modified ET-MCs in a fibrotic environment, a co-culture model was designed and the proliferative and migration ability, as well as the metabolic activity were evaluated.

Commercially available hypertrophic fibroblasts from idiopathic pulmonary fibrosis were cultured with the modified ET-MCs (empty viral vector or expressing DCN A). The co-culture was carried out in order to observe the effect on the proliferative and pro-fibrotic phenotype associated with them, and to evaluate the functionality of the TGFβ decoy released on cellular behavior such as cell proliferation, migration and metabolic activity.

The DCN A protein, produced by the modified ET-MCs, interferes with the proliferation of the fibrotic fibroblasts, with their migration, a fundamental event in the fibrotic process in vivo, but it also interferes with their metabolic activity.

Furthermore, surprisingly, the blocking of TGF-β does not cause damage at the level of the MCs that are known to use TGF-β as a proliferative factor, causing no damage to the performance of the MCs ex vivo.

The person of skill will understand that the method to produce MCs modified with a modifying agent described in the present invention and the modified MCs obtained with the method can also be used in treatments of other types of fibrosis, such as, as a non-limiting example, renal, cardiac, hepatic or pulmonary fibrosis, or that which occurs on other tissues and organs such as joints, bone marrow, the brain, eyes, the intestine, the peritoneum and retroperitoneum, the pancreas and skin.

In practice it has been found that the invention achieves the intended purposes. The invention as conceived is susceptible of modifications and variants, all of which come within the inventive concept. Furthermore, all the details can be replaced with other technically equivalent elements. In the practical implementation, the materials used, as well as their shapes and sizes, can be any whatsoever, depending on requirements, without thereby departing from the scope of protection.

1. Mesenchymal cells modified with a modifying agent for use in the treatment of fibrosis of lungs or kidney, wherein said modifying agent comprises a viral vector that codes for Decorin infecting said mesenchymal cells and that the infected mesenchymal cells express Decorin.

2. Mesenchymal cells according to claim 1, wherein said Decorin is Decorin isoform A, or isoform B, or isoform C, or isoform D or isoform E.

3. Mesenchymal cells according to claim 2, wherein said Decorin is Decorin isoform A.

4. Mesenchymal cells according to claim 1, wherein said mesenchymal cells are selected from mesenchymal cells of human or animal origin.

5. Mesenchymal cells according to claim 1, wherein said mesenchymal cells are selected from autologous mesenchymal cells or allogeneic mesenchymal cells.

6. Mesenchymal cells according to claim 1, wherein said mesenchymal cells are selected from mesenchymal cells originating from adipose tissue, or from bone marrow, from endometrial tissue, from placental tissue, from peripheral blood, from umbilical cord blood, from amniotic fluid and/or derivatives.

7. Mesenchymal cells according to claim 1, wherein said mesenchymal cells originate from endometrial tissue.

8. Mesenchymal cells according to claim 1, wherein said Decorin expressed by said modified mesenchymal cells is Decorin expressed repetitively.

9. Mesenchymal cells according to claim 1, wherein said viral vector is a lentivirus or a retrovirus.

10. A medicament for preventing or slowing down the fibrotic process of a kidney, the medicament comprising the
mesenchymal cells according to claim 1,
wherein the viral vector is lentiviral or retroviral infecting said modified mesenchymal cells comprising modified cells expressing Decorin.

11. A medicament for preventing or slowing down the fibrotic process of a lung, the medicament comprising the mesenchymal cells according to claim 1,
wherein the viral vector is lentiviral or retroviral infecting the mesenchymal cells comprising modified cells expressing Decorin.

12. A method for producing the mesenchymal cells according to claim 1, comprising the step of obtaining the mesenchymal cells, wherein the viral vector stably codes for a protein, and wherein the protein is Decorin, wherein the mesenchymal cells express said Decorin.

13. The method according to claim 12, wherein said modification comprises infecting said mesenchymal cells with said viral vector.

14. The method according to claim 12, wherein said Decorin is selected from Decorin isoform A, or isoform B, or isoform C, or isoform D, or isoform E.

15. The method according to claim 14, wherein said Decorin is Decorin isoform A.

16. The method according to claim 12, wherein said viral vector is selected from a lentivirus or a retrovirus.

17. The method according to claim 12, wherein said mesenchymal cells express HLA-G molecule.
